(19) United States
(12) Patent Application Publication
(10) Pub. No.: US 2006/0171968 A1
(43) Pub. Date: Aug. 3, 2006

(54) Method of preventive treatment of allergy by oromucosal administration of an allergy vaccine

(75) Inventors: Jens Brimnes, Herlev (DK); Jens Kildsgaard, Holte (DK)

Correspondence Address: BIRCH STEWART KOLASCH & BIRCH, PO BOX 747, FALLS CHURCH, VA, 22040-0747, US

(73) Assignee: Alk-Abello A/S, Horsholm (DK)

(21) Appl. No.: 11/325,451

(22) Filed: Jan. 5, 2006

Related U.S. Application Data
(60) Provisional application No. 60/641,729, filed on Jan. 7, 2005.

(30) Foreign Application Priority Data
Jan. 7, 2005 (DK) PA 2005 00040

(51) Int. Cl. A61K 39/35

(52) U.S. Cl. 424/275.1

(57) ABSTRACT

The present invention relates to a method of preventive treatment of allergy to an allergen in a subject comprising a) administering an allergy vaccine containing the allergen as active substance to the subject via an oromucosal route, b) wherein the subject to be treated is unsensitised in the sense of exhibiting no lgE response specific to the allergen, c) wherein the subject to be treated is free of clinical symptoms of any allergy, and d) wherein the preventive treatment is aimed at preventing or reducing subsequent clinical symptoms of the allergy associated with the allergen.

### TECHNICAL FIELD

The present invention relates to a method of preventive treatment of allergy to an allergen in a subject.

### BACKGROUND OF THE INVENTION

Allergy is a major health problem in countries where Western lifestyle is adapted. Furthermore, the prevalence of allergic disease is increasing in these countries. Although allergy in general may not be considered a life-threatening disease, asthma annually causes a significant number of deaths. An exceptional prevalence of about 30% in teenagers conveys a substantial loss in quality of life, working days and money, and warrants a classification among major health problems in the Western world.

Allergy is a complex disease. Many factors contribute to the sensitisation event. Among these is the susceptibility of the individual defined by an as yet insufficiently understood interplay between several genes. Another important factor is allergen exposure above certain thresholds. Several environmental factors may be important in the sensitisation process including pollution, childhood infections, parasite infections, intestinal microorganisms, etc. Once an individual is sensitised and the allergic immune response established, the presence of only minute amounts of allergen is efficiently translated into symptoms.

The natural course of allergic disease is usually accompanied by aggravation at two levels. Firstly, a progression of symptoms and disease severity, as well as disease progression, for example from hay fever to asthma.

Secondly, dissemination in offending allergens most often occurs resulting in allergic multi-reactivity. Chronic inflammation leads to a general weakening of the mucosal defense mechanisms resulting in unspecific irritation and eventually destruction of the mucosal tissue. Infants may become sensitised primarily to foods, i.e. milk, resulting in eczema or gastrointestinal disorders; however, most often they outgrow these symptoms spontaneously. These infants are at risk of developing inhalation allergy later in their lives.

The most important allergen sources are found among the most prevalent particles of a certain size in the air we breathe. These sources are remarkably universal and include grass pollens and house dust mite faecal particles, which together are responsible for approximately 50% of all allergies. Of global importance are also animal dander, i.e. cat and dog dander, other pollens, such as mugwort pollens, and micro-fungi, such as Alternaria. On a regional basis yet other pollens may dominate, such as birch pollen in Northern and Central Europe, ragweed in the Eastern and Central United States, and Japanese cedar pollen in Japan. Insects, i.e. bee and wasp venoms, and foods each account for approximately 2% of all allergies.

Allergy, i.e. type I hyper-sensitivity, is caused by an inappropriate immunological reaction to foreign non-pathogenic substances. Important clinical manifestations of allergy include asthma, hay fever, eczema, and gastro intestinal disorders. The allergic reaction is prompt and peaks within 20 minutes upon contact with the offending allergen. Furthermore, the allergic reaction is specific in the sense that a particular individual is sensitised to particular allergen(s), whereas the individual does not necessarily show an allergic reaction to other substances known to cause allergic disease. The allergic phenotype is characterized by a pronounced inflammation of the mucosa of the target organ and by the presence of allergen specific antibody of the lgE class in the circulation and on the surfaced of mast-cells and basophils.

An allergic attack is initiated by the reaction of the foreign allergen with allergen specific lgE antibodies, when the antibodies are bound to high affinity lgE specific receptors on the surface of mast-cells and basophils. The mast-cells and basophils contain preformed mediators, i.e. histamine, tryptase, and other substances, which are released upon cross-linking of two or more receptor-bound lgE antibodies. lgE antibodies are cross-linked by the simultaneous binding of one allergen molecule. It therefore follows that a foreign substance having only one antibody binding epitope does not initiate an allergic reaction. The cross-linking of receptor bound lgE on the surface of mast-cells also leads to release of signaling molecules responsible for the attraction of eosinophils, allergen specific T-cells, and other types of cells to the site of the allergic response. These cells in interplay with allergen, lgE and effector cells, lead to a renewed flash of symptoms occurring b 12 -24 hours after allergen encounter (late phase reaction).

Allergy disease management comprises diagnosis and treatment including prophylactic treatments. Diagnosis of allergy is concerned with by the demonstration of allergen specific lgE and identification of the allergen source. In many cases a careful anamnesis may be sufficient for the diagnosis of allergy and for the identification of the offending allergen source material. Most often, however, the diagnosis is supported by objective measures, such as skin prick test, blood test, or provocation test.

The therapeutic options fall in three major categories. The first opportunity is allergen avoidance or reduction of the exposure. Whereas allergen avoidance is obvious e.g. in the case of food allergens, it may be difficult or expensive, as for house dust mite allergens, or it may be impossible, as for pollen allergens. The second and most widely used therapeutic option is the prescription of classical symptomatic drugs like anti-histamines and steroids. Symptomatic drugs are safe and efficient; however, they do not alter the natural cause of the disease, neither do they control the disease dissemination. The third therapeutic alternative is specific allergy vaccination that in most cases reduces or alleviates the allergic symptoms caused by the allergen in question.

Conventional specific allergy vaccination is a causal treatment for allergic disease. It interferes with basic immunological mechanisms resulting in persistent improvement of the patients' immune status. Thus, the protective effect of specific allergy vaccination extends beyond the treatment period in contrast to symptomatic drug treatment. Some patients receiving the treatment are cured, and in addition, most patients experience a relief in disease severity and symptoms experienced, or at least an arrest in disease aggravation. Thus, specific allergy vaccination has preventive effects reducing the risk of hay fever developing into asthma, and reducing the risk of developing new sensitivities.

The immunological mechanism underlying successful allergy vaccination is not known in detail. A specific immune response, such as the production of antibodies against a particular pathogen, is known as an adaptive immune response. This response can be distinguished from the innate immune response, which is an unspecific reaction towards pathogens. An allergy vaccine is bound to address the adaptive immune response, which includes cells and molecules with antigen specificity, such as T-cells and the antibody producing B-cells. B-cells cannot mature into antibody producing cells without help from T-cells of the corresponding specificity. T-cells that participate in the stimulation of allergic immune responses are primarily of the Th2 type. Establishment of a new balance between Th1 and Th2 cells has been proposed to be beneficial and central to the immunological mechanism of specific allergy vaccination. Whether this is brought about by a reduction in Th2 cells, a shift from Th2 to Th1 cells, or an up-regulation of Th1 cells is controversial. Recently, regulatory T-cells have been proposed to be important for the mechanism of allergy vaccination. According to this model regulatory T-cells, i.e. Th3 or Tr1 cells, down-regulate both Th1 and Th2 cells of the corresponding antigen specificity. In spite of these ambiguities it is generally believed that an active vaccine must have the capacity to stimulate allergen specific T-cells, preferably TH1 cells.

Specific allergy vaccination (SAV), formerly known as Specific Immunotheraphy or Hyposensitization, has been used for the treatment of Type 1 lgE mediated allergic disease since the beginning of this century.

The general benefits obtained through SAV are: a) reduction of allergic symptoms and medicine consumption, b) improved tolerance towards the allergens in the eyes, nose and lungs and c) reduced skin reactivity (early and late phase reactions).

The basic mechanism behind the improvement obtained by SAV is unknown, but a number of common features can be extracted from the numerous SAV studies performed in the last decades: 1) the amount of total lgE is unchanged during the treatment period, 2) the amount of allergen specific lgE increases transiently during updosing, then it falls back to the initial (pretreatment) level, 3) the epitope specificity and affinity of lgE remains unchanged, 4) allergen specific lgG, in particularly lgG4, raises sharply during SAV, 5) a new Th0/1/Reg response is apparently initiated and 6) the Th2 response seem unchanged. There is no correlation between the effect induced by SAV and the onset of specific lgG.

SAV induces a new immune response which matures during the treatment period (Th0/1 T-cells are recruited, an allergen specific lgX (X may be A1, A2, G1, G2, G3, G4, M or D) is initiated). As the affinity (or amount/affinity) of the new antibody response, lgX, has matured, lgX may compete efficiently with lgE for the allergen(s), inhibiting the “normal” Th2 based allergic response characterised by the cross-linking of receptor bound lgE on the surface of mast-cells and basophils. Hence, clinical symptoms will gradually be reduced.

Specific allergy vaccination is, in spite of its virtues, not in widespread use, primarily for two reasons. One reason is the inconveniences associated with the traditional vaccination programme that comprises repeated vaccinations i.a. injections over a several months. The other reason is, more importantly, the risk of allergic side reactions. Ordinary vaccinations against infectious agents are efficiently performed using a single or a few high dose immunizations. This strategy, however, cannot be used for allergy vaccination since a pathological immune response is already ongoing.

Conventional specific allergy vaccination is therefore carried out using multiple subcutaneous immunizations applied over an extended time period. The course is divided in two phases, the up dosing and the maintenance phase. In the up dosing phase increasing doses are applied, typically over a 16-week period, starting with minute doses. When the recommended maintenance dose is reached, this dose is applied for the maintenance phase, typically with injections every six weeks. Following each injection the patient must remain under medical attendance for 30 minutes due to the risk of anaphylactic side reactions, which in principle although extremely rare could be life-threatening. In addition, the clinic should be equipped to support emergency treatment. There is no doubt that a vaccine based on a different route of administration would eliminate or reduce the risk for allergic side reactions inherent in the current subcutaneous based vaccine as well as would facilitate a more widespread use, possibly even enabling self vaccination at home.

Attempts to improve vaccines for specific allergy vaccination have been performed for over 30 years and include multifarious approaches. Several approaches have addressed the allergen itself through modification of the lgE reactivity.

Holt et al. (“Suppression of lgE responses following inhalation of antigen”, Immunology Today, vol. 8, No. 1,1987) mentions the fact that as a response to exposure to inhaled or oronasally instilled allergen, tolerance is induced in the upper respiratory tract corresponding to that induced in the gastrointestinal tract by dietary antigens.

Holt et al. (“Sublingual allergen administration. I. Selective suppression of lgE production in rats by high allergen doses”, Clinical Allergy, 1988, Volume 18, pages 229-234) relates to sublingual administration of an allergen (ovalbumin) to naive rats for seven consecutive days followed by a parenteral challenge by ovalbumin five days after the last sublingual dose. The results showed a selective suppression of lgE specific to ovalbumin. It is speculated that the mechanism of the treatment involves stimulation of allergen-specific suppressor cells. It is further mentioned that the treatment proposed involves naive animals and should be distinguished from a sublingual desensitisation process.

WO 95/17208 discloses a method of prevention of allergic disease comprising administering to a previously unsensitised subject a dose of allergen effective to induce establishment of a stable population of allergen-specific T-helper-1-like memory lymphocytes capable of inhibiting activity of allergen-specific T-helper-2-like lymphocytes. The subject to be treated is preferably between 3 months and 7 years. As allergen e.g. house dust mites, grass pollen and tree pollen are mentioned. The administration of the allergen may be carried out by the oral, intranasal, oronasal, rectal, intradermal, intramuscular or subcutaneous route.

The home page www.immunetolerance.org (11 Oct. 2004) discloses e.g. a planned clinical study of preventive treatment of children without sensitisation to inhalants, wherein sublingual drops containing either allergen (house dust mite, timothy grass and cat) are administered to the children, and wherein the children are followed for the development of allergy for three years. The children recruited for the study have a history of atopic dermatitis or food allergy and their biological mother or father or one sibling has a history of atopy.

The object of the present invention is to provide an improved method of preventive treatment of individuals, in particular children.

### SUMMARY OF THE INVENTION

This object is obtained with the present invention, which relates to a method of preventive treatment of allergy to an allergen in a subject comprising
a) administering an allergy vaccine containing the allergen as active substance to the subject via an oromucosal route,
b) wherein the subject to be treated is unsensitised in the sense of exhibiting no lgE response specific to the allergen,
c) wherein the subject to be treated is free of clinical symptoms of any allergy, and
d) wherein the preventive treatment is aimed at preventing or reducing subsequent clinical symptoms of the allergy associated with the allergen.

The present invention is based on the surprising finding that the effect of preventive treatment, wherein the administration is carried out via the oromucosal route is far more effective than preventive treatment, wherein the administration is carried out via other mucosal routes.

It is believed that the mechanism involved in prevention of an allergy is induction of oral tolerance corresponding to that induced in the gastrointestinal tract by dietary antigens. It is further believed that preventive treatment is most effective when carried out before sensitisation, i.e. before the immune system response begins to shift toward an allergic Th2 cell response. In other words it is in general advantageous to treat children as young as possible in the sense that the younger the child is the higher is the chance that it has not yet been exposed to an allergen. Also, it is believed that due to the effectiveness of such early preventive treatment, treatment may be effected with smaller doses, fewer administrations and/or a shorter period of treatment compared to specific allergy vaccination of adults with developed clinical symptoms. Due to the mildness of the protocol of the preventive treatment, it is suitable for use in general vaccination programs of all children or large groups of selected children.

The invention further relates to the use of an allergen for the manufacture of a vaccine for the preventive treatment of allergy in a subject,
a) wherein the vaccine is suitable for administration via an oromucosal route,
b) wherein the subject to be treated is unsensitised in the sense of exhibiting no lgE response specific to the allergen,
c) wherein the subject to be treated is free of clinical symptoms of any allergy, and
d) wherein the preventive treatment is aimed at preventing or reducing subsequent clinical symptoms of the allergy associated with the allergen.

### SHORT DESCRIPTION OF THE FIGURES

FIG. 1A-C show serum levels of Phl p specific total lgG, lgG1 and lgG2 a in mice that have been treated with SLIT for six weeks.

FIG. 1 D-F show serum levels of Phl p specific total lgG, lgG1 and lgG2a in mice subjected to an identical administration of SLIT followed by one i.p. immunisation with Phl p extract (5 kSQ/alum).

FIG. 2A shows serum levels of Phl p specific lgE in mice that have been treated with SLIT for six weeks.

FIG. 2B shows serum levels of Phl p specific lgE in mice that have been treated with SLIT followed by one i.p. immunisation with Phl p extract.

FIG. 3 shows Phl p-specific lgE levels in sera of SLIT treated (hatched lines) and buffer treated control mice (solid lines).

FIG. 4 shows Phl p-specific lgA levels in BAL of SLIT treated and buffer treated control mice.

FIG. 5 shows the proliferation of spleen cells from mice treated with Phl p SLIT.

FIG. 6A and 6B show the proliferation and cytokine production, respectively, of spleen cells from mice treated with Phl p SLIT followed by one immunization.

FIG. 7A and 7B show the proliferation of spleen cells from mice treated with Phl p SLIT for three and six weeks, respectively, followed by one immunization.

FIG. 8 shows the proliferation of spleen cells from mice treated with different doses of Phl p SLIT followed by one immunization.

### DETAILED DESCRIPTION OF THE INVENTION

### Allergen

The allergen of the formulation according to the present invention may be any naturally occurring protein that has been reported to induce allergic, i.e. lgE mediated, reactions upon their repeated exposure to an individual. Examples of naturally occurring allergens include pollen allergens (tree-, herb, weed-, and grass pollen allergens), insect allergens (inhalant, saliva and venom allergens, e.g. mite allergens, cockroach and midges allergens, hymenopthera venom allergens), animal hair and dandruff allergens (from e.g. dog, cat, horse, rat, mouse etc.), and food allergens. Important pollen allergens from trees, grasses and herbs are such originating from the taxonomic orders of Fagales, Oleales, Pinales and platanaceae including i.a. birch (Betula), alder (Alnus), hazel (Corylus), hornbeam (Carpinus) and olive (Olea), cedar (Cryptomeriaand Juniperus), Plane tree (Platanus), the order of Poales including i.a. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other important inhalation allergens are those from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (superfamily Apidae), wasps (superfamily Vespidea), and ants (superfamily Formicoidae). Important inhalation allergens from fungi are i.a. such originating from the genera Alternaria and Cladosporium.

In a particular embodiment of the invention the allergen is Bet v 1, Aln g 1, Cor a 1 and Car b 1, Que a 1, Cry j 1, Cry j 2, Cup a 1, Cup s 1, Jun a 1, Jun a 2, jun a 3, Ole e 1, Lig v 1, Pla l 1, Pla a 2, Amb a 1, Amb a 2, Amb t 5, Art v 1, Art v 2 Par j 1, Par j 2, Par j 3, Sal k 1, Ave e 1, Cyn d 1, Cyn d 7, Dac g 1, Fes p 1, Hol l 1, Lol p 1 and 5, Pha a 1, Pas n 1, Phl p 1, Phl p 5, Ph p 6, Poa p 1, Poa p 5, Sec c 1, Sec c 5, Sor h 1, Der f 1, Der f 2, Der p 1, Der p 2 ,, Der p 7, Der m 1, Eur m 2, Gly d 1, Lep d 2, Blo t 1, Tyr p 2, Bla g 1, Bla g 2, Per a 1, Fel d 1, Can f 1, Can f 2, Bos d 2, Equ c 1, Equ c 2, Equ c 3, Mus m 1, Rat n 1, Apis m 1, Api m 2, Ves v 1, Ves v 2, Ves v 5, Dol m 1, Dil m 2, Dol m 5, Pol a 1, Pol a 2, Pol a 5, Sol i 1, Sol i 2, Sol i 3 and Sol i 4, Alt a 1, Cla h 1, Asp f 1, Bos d 4, Mal d 1, Gly m 1, Gly m 2, Gly m 3, Ara h 1, Ara h 2, Ara h 3, Ara h 4, Ara h 5 or shufflant hybrids from Molecular Breeding of any of these.

In a preferred embodiment of the invention the allergen is selected from the group consisting of a tree pollen allergen, a grass pollen allergen, a dust mite allergen, a herb allergen and an animal allergen. Preferably, the allergen is selected from the group consisting of a grass pollen allergen, a dust mite allergen, a ragweed allergen, a cedar pollen, a cat allergen and a birch allergen.

In yet another embodiment of the invention the formulation comprises at least two different types of allergens either originating from the same allergic source or originating from different allergenic sources e.g. grass group 1 and grass group 5 allergens or mite group 1 and group 2 allergens from different mite and grass species respectively, weed antigens like short and giant ragweed allergens, different fungis allergens like alternaria and cladosporium, tree allergens like birch, hazel, hornbeam, oak and alder allergens, food allergens like peanut, soybean and milk allergens .

The allergen incorporated into the formulation may be in the form of an extract, a purified allergen, a modified allergen, a recombinant allergen or a mutant of a recombinant allergen. An allergenic extract may naturally contain one or more isoforms of the same allergen, whereas a recombinant allergen typically only represents one isoform of an allergen. In a preferred embodiment the allergen is in the form of an extract. In another preferred embodiment the allergen is a recombinant allergen. In a further preferred embodiment the allergen is a naturally occurring low lgE-binding mutant or a recombinant low lgE-binding mutant.

Allergens may be present in equi-molar amounts or the ratio of the allergens present may vary preferably up to 1:20.

In a further embodiment of the invention the low lgE binding allergen is an allergen according to WO 99/47680, WO 02/40676 or WO 03/096869 A2.

### Preventive Treatment

The mechanisms of the preventive treatment carried out in the present invention are not fully investigated or understood.

It has been speculated that it is preferable to carry out a mucosal administration of a vaccine via the mucosa, which is subject to the natural exposure to the antigenic agent. Accordingly, for allergies to airborne mucosal antigenic agents, it is preferred to use administration via the respiratory system, preferably an oromucosal administration.

Oromucosal administration includes buccal and sublingual administration. The oromucosal administration may be carried out using any available oromucosal administration formulation, including a spray, an aerosol, a mixture, a suspension, a dispersion, an emulsion, a gel, a paste, a syrup, a cream, an ointment, a solution, fast dispersing dosage forms, drops and lozenges.

In a preferred embodiment of the invention, sublingual immunotherapy (SLIT) is used, in which case fast dispersing dosage forms, drops and lozenges are preferred formulations.

Examples of fast dispersing dosage forms are those disclosed in U.S. Pat. No. 5,648,093, WO 00/51568, WO 02/13858, W099/21579, WO 00/44351, U.S. Pat. No. 4,371,516 and EP-278 877, as well as co-pending DK PA 2003 00279 and DK PA 2003 00318 filed in the assignee name of ALK-Abelló A/S. Preferred fast dispersing dosage forms are those produced by freeze-drying. Preferred matrix forming agents are fish gelatine and modified starch.

Classical incremental dosage desensitisation, where the dose of allergen in the form of a fast dispersing solid dosage form is increased to a certain maximum, may be used in the present invention. The preferred potency of a unit dose of the dosage form is from 150-1000000 SQ-u/dosage form, more preferred the potency is from 500-500000 SQ-u/dosage form and more preferably the potency is from 1000-250000 SQ-u/dosage form, even more preferred 1500-125000 SQ-u/dosage form most preferable 1500-75000 SQ-u/dosage form.

In another embodiment of the invention the dosage form is a repeated mono-dose, preferably within the range of 1500-75000 S′Q-u/dosage form.

In one embodiment of the invention, the subject is subjected to a vaccination protocol comprising daily administration of the vaccine. In another embodiment of the invention the vaccination protocol comprises administration of the vaccine every second day, every third day or every fourth day. For instance, the vaccination protocol comprises administration of the vaccine for a period of more than 4 weeks, preferably more than 8 weeks, more preferably more than 12 weeks, more preferably more than 16 weeks, more preferably more than 20 weeks, more preferably more than 24 weeks, more preferably more than 30 and most preferably more than 36 weeks.

The period of administration may a continuous period. Alternatively, the period of administration is a discontinuous period interrupted by one or more periods of non-administration. Preferably, the (total) period of non-administration is shorter than the (total) period of administration.

In a further embodiment of the invention, the vaccine is administered to the subject once a day. Alternatively, the vaccine is administered to the subject twice a day. The vaccine may be a uni-dose vaccine.

The subject to be treated is a mammal in need of preventive treatment, preferably a mammal selected from the group consisting of humans, cats and dogs, in particular humans.

### Sensitisation

The subject to be treated is unsensitised in the sense of exhibiting no lgE response specific to the allergen administered. In connection with the present invention the expression “exhibiting no lgE response specific to the allergen” means a level of allergen-specific lgE antibody undetectable in a conventional immunoassay. The level of allergen-specific lgE antibody may be determined using any conventional immunoassay, e.g. those described in WO 94/11734 and WO 99/67642 and the lgE immunoassay described in Example 1 of the present application.

In a particular embodiment of the invention the subject is unsensitised in the sense of exhibiting no Th2 cell response specific to the allergen.

In a particular embodiment of the invention the subject is unsensitised in the sense of exhibiting no positive allergen-specific response in a Skin Prick Test (SPT).

In a further particular embodiment of the invention, the subject is unsensitised to any allergen.

In a further particular embodiment of the invention the subject is less than 40 years, preferably less than 30 years, more preferably less than 20 years and most preferably between ½ and 10 years of age. Preferably, the subject is treated before its first exposure to the allergen, e.g. before the first pollen season, to eliminate the risk that the subject is sensitised.

As mentioned above it is in general advantageous to treat children as young as possible in the sense that the younger the child is the higher is the chance that it has not yet been exposed to an allergen. However, the age of the child to be treated should be selected with due consideration to the fact that exposure to allergen in the very early phase of infancy does involve the risk of priming the child for subsequent pathogenic T-cell reactivity as opposed to inducing protective tolerance. This is consistent with the existence of an early period of high risk for allergic sensitisation, presumably due to delayed postnatal maturation of the immune system in the child. In accordance with this it is known that a characteristic sequence of manifestations, which is often observed in children during the first decade of life, involves 1) atopic dermatitis, which becomes manifest during the first months of life and may persist for months, years or decades, 2) infantile wheeze, which may develop into bronchial asthma, and 3) intermittent or persistent allergic rhinitis, which is extremely rare during the first two years of life, whereas from the third year on the prevalence increases to more than 20% at the end of the first decade.

### Clinical Symptoms

The subject to be treated is free of clinical symptoms of any allergy, i.e. the clinical symptoms of allergy associated with any allergen.

The clinical symptoms of allergy may be any conventional symptom, including rhinitis, conjunctivitis, rhinorrhea, nasal obstruction, sinusitis, sneezing, atopic dermatitis, itching, watery eyes, watery nose, wheezing and skin irritation.

A number of factors are indicative for development of allergy with manifested clinical symptoms later in life. One such indicating factor is the presence of one or more allergies in one or both parents or grandparents or in one or more sibling. The preventive treatment according to the invention is particularly suitable for subjects exhibiting the said indicating factor. However, the subject to be treated may also be a subject, who does not exhibit the said indicating factor.

### Formulation of Allerqy Vaccine

The allergy vaccine used in the method of the invention may be in the form of any formulation suitable for administration to an oromucosal surface, including formulations selected form the group consisting of a spray, an aerosol, a mixture, tablets, capsule (hard and soft), a suspension, a dispersion, granules, a powder, a solution, an emulsion, chewable tablets, drops, a gel, a paste, a syrup, a cream, a losenge (powder, granulate, tablets), a fast-dispersing dosage form, a gas, a vapour, an ointment and a stick.

It is to be understood that the vaccine of the invention may further comprise additional adjuvants and other excipients suitable for such type of formulation. Such additional adjuvants and excipients are well-known to the person skilled in the art and include i.a. solvents, emulsifiers, wetting agents, plasticizers, colouring substances, fillers, preservatives, viscosity adjusting agents, buffering agents, mucoadhesive substances, and the like. Examples of formulation strategies are well-known to the person skilled in the art.

The mucosal allergy vaccine may include an adjuvant, which may be any conventional adjuvant, including oxygen-containing metal salts, heat-labile enterotoxin (LT), cholera toxin (CT), cholera toxin B subunit (CTB), polymerised liposomes, mutant toxins, e.g. LTK63 and LTR72, microcapsules, interleukins (e.g. IL-1β, IL-2, IL-7, IL-12, INFγ), GM-CSF, MDF derivatives, CpG oligonucleotides, LPS, MPL, phosphophazenes, Adju-Phos®, glucan, antigen formulation, liposomes, DDE, DHEA, DMPC, DMPG, DOC/Alum Complex, Freund's incomplete adjuvant, ISCOMs®, LT Oral Adjuvant, muramyl dipeptide, monophosphoryl lipid A, muramyl tripeptide, and phospatidylethanolamine.

The oxygen-containing metal salt may be any oxygen-containing metal salt providing the desired effect. In a preferred embodiment, the cation of the oxygen-containing metal salt is selected from Al, K, Ca, Mg, Zn, Ba, Na, Li, B, Be, Fe, Si, Co, Cu, Ni, Ag, Au, and Cr. In a preferred embodiment, the anion of the oxygen-containing metal salt is selected from sulphates, hydroxides, phosphates, nitrates, iodates, bromates, carbonates, hydrates, acetates, citrates, oxalates, and tartrates, and mixed forms thereof. Examples are aluminium hydroxide, aluminium phosphate, aluminium sulphate, potassium aluminium sulphate, calcium phosphate, Maalox (mixture of aluminium hydroxide and magnesium hydroxide), beryllium hydroxide, zinc hydroxide, zinc carbonate, zinc chloride, and barium sulphate.

Allergy vaccines in the form of an aqueous solution or a fast-dispersing tablet, cf. WO 04/047794, are particularly suitable for buccal and sublingual administration.

### Administration Via the Parenteral Route

In a preferred embodiment of the invention, the method comprises administering an allergy vaccine containing the allergen as active substance to the subject via a parenteral route. The administration via the parenteral route is carried out in addition to the administration via the oromucosal route and serves to increase the effect of the preventive treatment. It is believed that by using two different administration routes, a boosting, i.e. synergistic, effect in increasing the effect of the preventive treatment is obtained.

In a particular embodiment of the invention the administration via a parenteral route is carried out subsequent to the administration via the oromucosal route. In another particular embodiment of the invention the administration via a parenteral route is carried out prior to the administration via the oromucosal route.

### Definitions

In connection with the present invention the following definitions are used:

The term “oromucosal administration” refers to a route of administration where the dosage form is placed under the tongue or anywhere else in the oral cavity (buccal administration) to allow the active ingredient to come in contact with the mucosa of the oral cavity or the pharynx of the patient in order to obtain a local or systemic effect of the active ingredient. An example of an oromucosal administration route is sublingual administration.

The term “sublingual administration” refers to a route of administration, where a dosage form is placed underneath the tongue in order to obtain a local or systemic effect of the active ingredient.

The term “SQ-u” means SQ-Unit: The SQ-Unit is determined in accordance with ALK-Abello A/S's “SQ biopotency”-standardisation method, where 100,000 SQ units equal the standard subcutaneous maintenance dose.

Normally 1 mg of extract contains between 100,000 and 1,000,000 SQ-Units, depending on the allergen source from which they originate and the manufacturing process used. The precise allergen amount can be determined by means of immunoassay i.e. total major allergen content and total allergen activity.

### EXAMPLES

### Example 1

Preventive treatment comprising sublingual administration and SAV by parenteral administration in mice

### Methods

Animals

Female, 6-10 week-old BALB/c mice were bred in-house and maintained on a defined diet not containing component cross reacting with antisera to Phl p. Each experimental group consisted of 8-10 animals.

Animal experiments

Naive mice received sublingual immunotherapy (SLIT) by buccal administration of Phl p (5 μl) daily for two to six weeks and at three different concentrations, including a buffer control. Following SLIT treatment, the mice were either sacrificed or immunized intraperitoneally (i.p.) one, two or three times with aluminiumhydroxide-adsorbed Phl p (week 6-9) and sacrificed 10 days after the last immunization. Following sacrifice blood, bronchoalveolar fluid (BAL), nasopharyngeal fluid (NAL), spleen and cervical lymph nodes were collected for analysis.

Using this protocol it is possible to see whether a SLIT treatment is able to prime the immune system so as to increase the effect of the subsequent intraperitoneal treatment.

lgA Assay

Estapore magnetic beads (Estapore IB-MR/0,86) coupled to goat a-mouse lgA are incubated with BAL or NAL. Then washing and incubation with biotinylated allergen is carried out. Then washing and incubation with streptavidin labeled LITE reagent is carried out, and after washing light luminescence is measured in a luminometer (Magic Lite Analyser EQ).

lgE assay

Estapore magnetic beads (Estapore IB-MR/0,86) coupled to a-mouse lgE A0201 are incubated with mouse serum. Then washing and incubation with biotinylated allergen is carried out. Then washing and incubation with streptavidin labeled LITE reagent is carried out, and after washing light luminescence is measured in a luminometer (Magic Lite Analyser EQ).

lgG, lgG1 and lgG2a Assay

1. Coating. 100 μl Phl p (10 μg/ml) extract is added to the wells of an ELISA plate (NUNC Maxisorp 439454). The plates are allowed to stand until the next day at 4-8° C.

2. Washing. The coated plates are washed with a buffer.

3. Blocking. 200 μl 2% Casein buffer is added to each well and incubated at room temperature for one hour on a shaking table. After incubation the Casein buffer is removed.

4. Serum. The serum sample is diluted, and 100 μl diluted sample is added to the well of a plate and incubated at room temperature for two hours on a shaking table.

5. Washing.

6. Conjugate. 100 μl biotinylated rabbit anti-mouse lgG/lgG1/lgG2a diluted in 0.5% BSA buffer is added to each well and allowed to stand at room temperature for one hour on a shaking table.

7. Washing.

8. 100 μl Streptavidin-HRP diluted in 0.5% BSA buffer is added to each well and allowed to stand at room temperature for one hour on a shaking table.

9. Substrate: 100 μl TMP (3,3′,5,5′-Tetramethylbenzidine, Kem-En-TecTMB ONE) is added to each well and incubated 20 min.

10. Stop. 100 μl 0.5 M H₂SO₄ is added to each well to stop the reaction.

11. Measurement. The resulting reaction mixtures are subjected to a spectrophotometric measurement at 450 nm endpoint (Bio Kinetics Reader EL-340).

T-cell proliferation assay

Spleens were teased into single cell suspension and washed three times in medium. Cells were counted and adjusted to 1.67×106 cells/mL. 3×10⁵ cells were added to each well of a 96 well flat-bottomed culture plate and the cells were stimulated by 0, 10 and 40 μg/mL Phleum pratense extract. The cells were cultured for 6 days at 37° C. and 5% CO₂. Proliferation was measured by adding 0.5 μCi of ³H-thymidine to each well for the last 18 hours of the culture period, followed by harvesting the cells and counting the incorporated radiolabel.

Cytokine Measurements

Spleens were teased into single cell suspension and washed three times in medium. Cells were counted and adjusted to 3×10⁶ cells/mL. 2,5×10⁶ cells were added to each well of a 24 well culture plate and the cells were stimulated by 0 and 40 μg/mL Phleum pratense extract. Supernatants, harvested at day 3 and day 6, were analyzed for the presence of IL-2, IL-4, IL-5, Interferon gamma and Tumor necrosis factor alpha using the cytometric bead array assay from Becton Dickinson. In brief, the above mentioned supernatants were mixed with fluorescent beads coated with cytokine specific capture antibodies as well as PE-conjugated, cytokine specific detection antibodies. After washing unbound material away the sample data were acquired using a flow cytometer.

### Results

Antibody Response

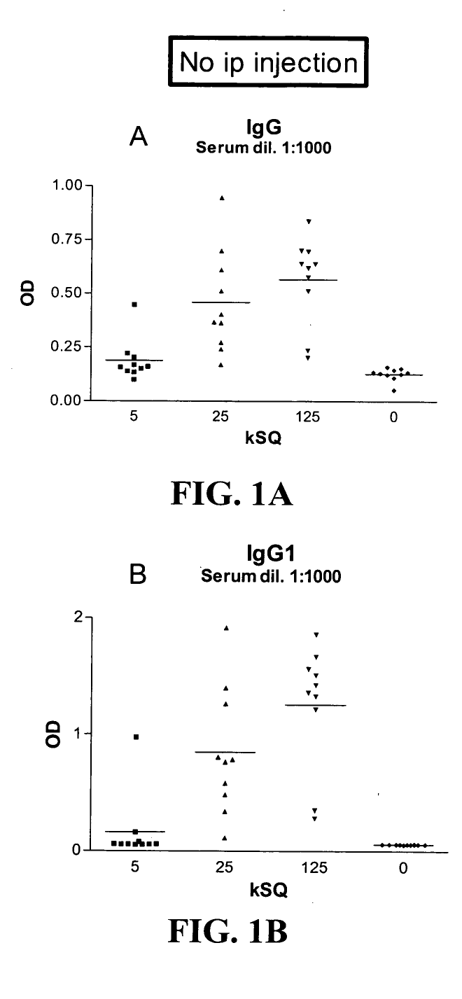

FIG. 1A-C show serum levels of Phl p specific total lgG, lgG1 and lgG2a in mice that have been treated with SLIT for six weeks. Each group of mice received daily SLIT doses of either 5, 25 or 125 kSQ, or buffer as a control. FIG. 1 D-F show serum levels of Phl p specific total lgG, lgG1 and lgG2a in mice subjected to an identical administration of SLIT followed by one i.p. immunisation with Phl p extract (5 kSQ/alum).

In the absence of i.p. injections sublingual administration of Phl p generated increasing levels of Phl p specific lgGs that were proportional to the time and dose of SLIT administration (FIG. 1A-C). Panels D-F show that SLIT followed by one i.p. injection generated lgG levels that were increased up to 40 times compared to no i.p. injection, demonstrating a priming or sensitizing effect by sublingual allergen administration. Furthermore, mice that received buffer alone as SLIT treatment does not generate significant amounts of antibodies to Phl p after one i.p. immunisation.

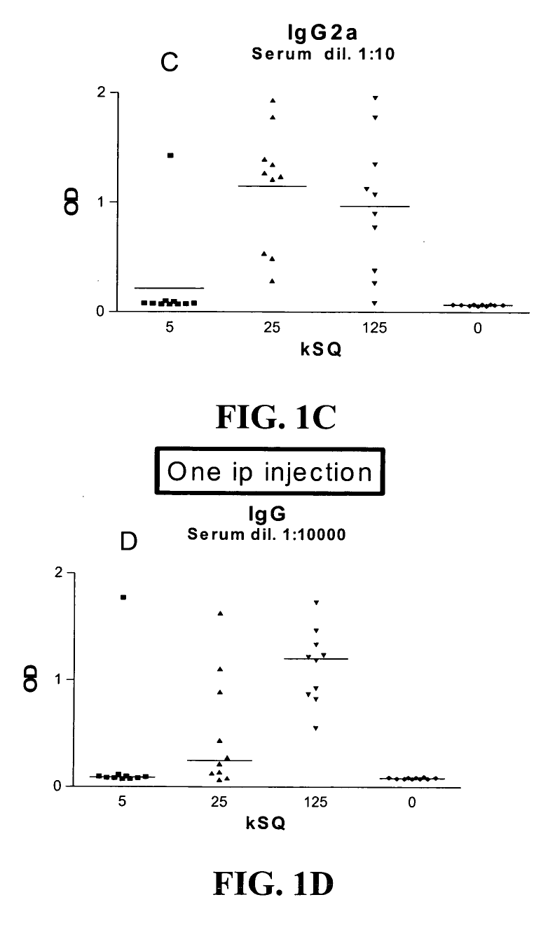

FIG. 2A shows serum levels of Phl p specific lgE in mice that have been treated with SLIT for six weeks. Each group of mice received daily SLIT doses of either 5, 25 or 125 kSQ Phl p extract, or buffer as control. FIG. 2B shows serum levels of Phl p specific lgE in mice that have been subjected to an identical administration of SLIT followed by one i.p. immunisation with Phl p extract (5 kSQ/alum). (RLU: Relative light units).

Prior to i.p. injections, increased levels of specific lgE, proportional to dose and time of SLIT treatment, were observed in serum. However, as for the lgG antibodies, one i.p. injection generated lgE levels that were increased up to 60 times in mice having received Phl p-SLIT (FIG. 13). Again, a single i.p. immunisation of mice treated with buffer-SLIT does not generate significant levels of lgE.

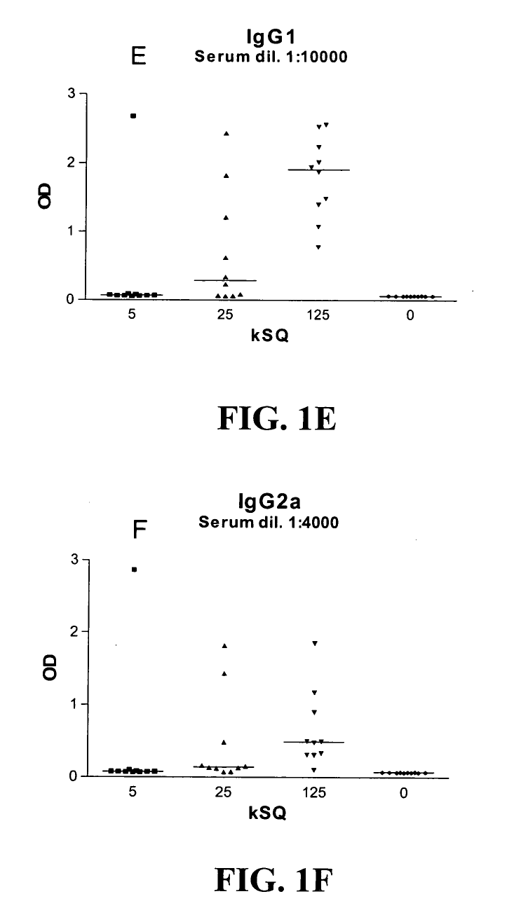

FIG. 3 shows Phl p-specific lgE levels in sera of SLIT treated (25 kSQ) (hatched lines) and buffer treated control mice (solid lines). Following SLIT treatment, the mice were immunised i.p. with Phl p extract (25 kSQ/alum) three times. One week after each immunisation the mice were bled and serum analysed for lgE levels. The first immunisation generated high lgE levels in mice having received Phl p-SLIT compared to control mice. The second and third immunisations generated increasing levels of specific lgE antibodies in the control mice whereas a strong down-regulation of the lgE-response is observed for the group of mice that received Phl p-SLIT.

The buccal administration of Phl p extract sensitises or primes the mice, since a single i.p. immunisation generates high and dose-dependent antibody levels. Although buccal administration of Phl p primes the mice as described above, repeated i.p. injections lead to a decrease in lgE levels, indicating that a specific suppression of the B cell response has been induced.

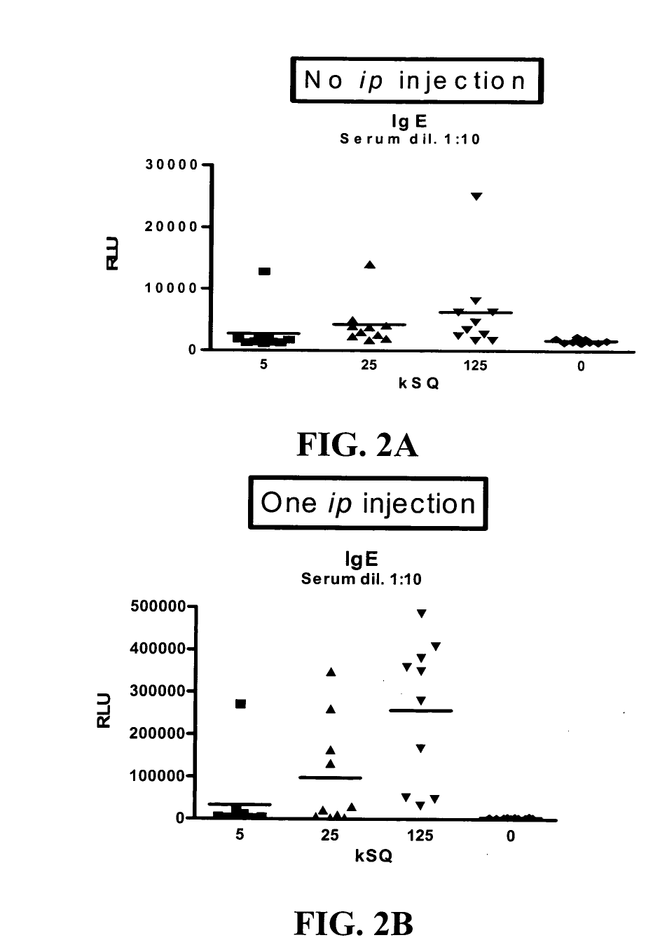

FIG. 4 shows Phl p-specific lgA levels in BAL of SLIT treated (25 kSQ) and buffer treated control mice. Following SLIT treatment, the mice were immunised i.p. with Phl p extract (25 kSQ/alum) three times. The lgA levels in BAL are significantly higher in Phl p-SLIT treated mice as compared to buffer-SLIT treated mice (P ≦0.05, Mann Whitney test). In contrast to the down-regulation of the lgE-response, specific lgA levels increased in BAL of mice treated with Phl p-SLIT after three i.p. immunisations.

T cell response

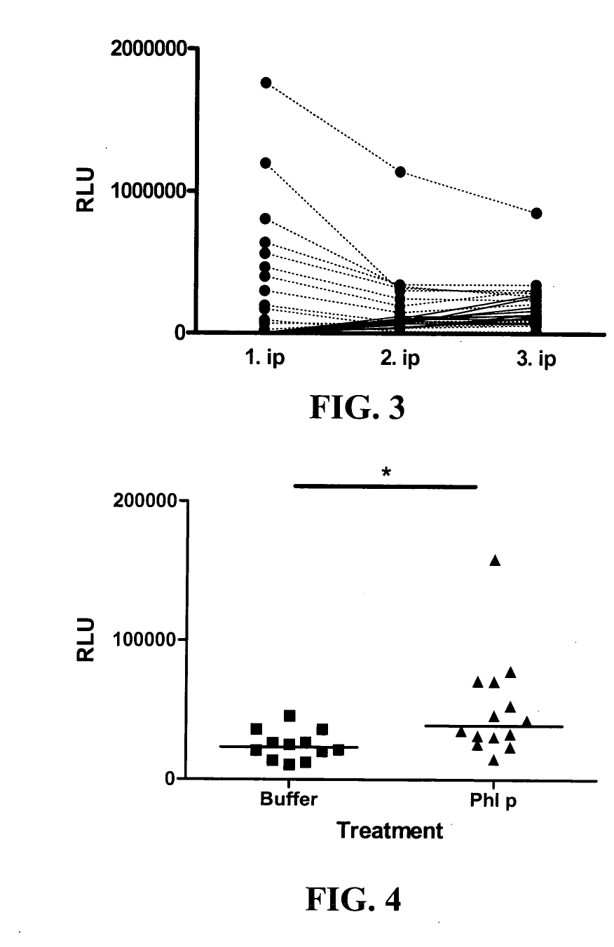

FIG. 5 shows the proliferation of spleen cells from mice treated with Phl p SLIT. Mice were given Phl p (25 kSQ) sublingually for either 2, 4 or 6 weeks. Following this, spleen cells were isolated and stimulated with Phl p in vitro at the indicated concentrations. Proliferation was measured after 6 days of incubation. As a control, spleen cells from immunised mice were included. Each column represents the mean of 6 individual mice and error bars indicate standard error of mean.

As seen in FIG. 5, SLIT given for either 2, 4 or 6 weeks did not lead to activation of spleen cells, upon allergen-specific restimulation in vitro, as the proliferation did not exceed the background values. As a positive control a strong proliferative response was seen in mice immunized with 0, 10 or 40 mcg Phl p/ml.

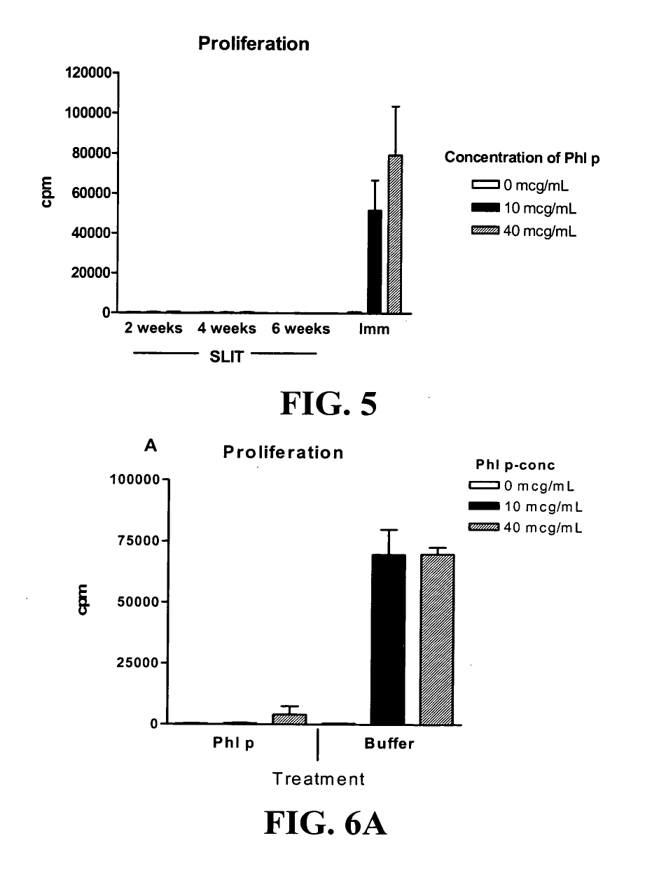

FIG. 6 shows the proliferation and cytokine production of spleen cells from mice treated with Phl p SLIT followed by one immunization. Mice were treated with either Phl p SLIT or buffer for 6 weeks, followed by one i.p. injection of alum-adsorbed Phl p. Spleen cells were isolated 8 days later and restimulated in vitro with Phl p. FIG. 6A: The proliferation measured after 6 days of incubation. FIG. 6B: Supernatants were harvested at day 5 and analyzed for TNF-α, IFN-γ, IL-4, IL-5 and IL-2. Each bar represents the mean of 8 individual mice. Error bars indicate standard error of mean.

As shown in FIG. 6, SLIT treatment with Phl p led to the induction of antigen-specific systemic tolerance, as the proliferation of spleen cells from mice that were treated with Phl p SLIT were dramatically reduced compared to mice that only received buffer. Similarly, the secretion of TNF-α, IFN-γ, IL-4 and IL-5 by the in vitro stimulated spleen cells was also reduced in mice that were treated with Phl p SLIT. IL-2 secretion was low in both SLIT and buffer treated mice.

Duration of SLIT Treatment

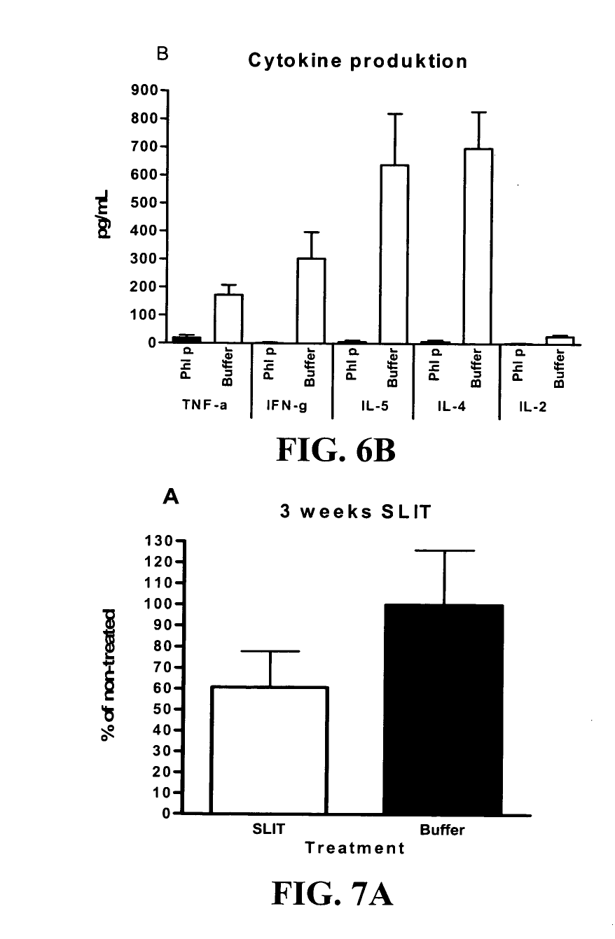

FIG. 7 shows the proliferation of spleen cells from mice treated with Phl p SLIT followed by one immunization. Mice were treated with Phl p SLIT for either 3 (FIG. 7A) or 6 (FIG. 7B) weeks followed by one i.p. injection of alum-adsorbed Phl p. Spleen cells were isolated 10 days later and restimulated in vitro with Phl p. Proliferation was measured after 6 days of incubation.

The duration of SLIT treatment seems to be important regarding the induction of T-cell tolerance. As seen in FIG. 7, SLIT-treatment for three weeks prior to immunization resulted in a less effective down-regulation of the proliferative response compared to six weeks of SLIT treatment.

### Dose Response:

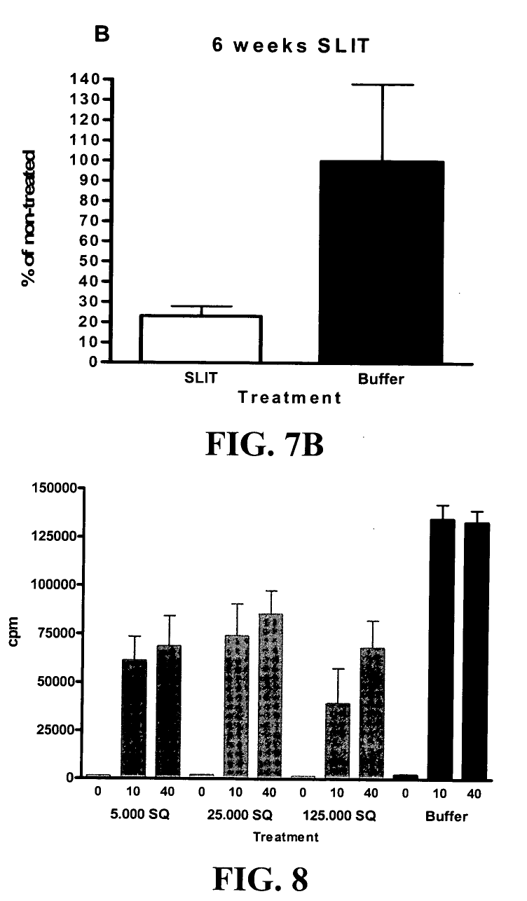

FIG. 8 shows the proliferation of spleen cells from mice treated with Phl p SLIT followed by one immunization. Mice were treated with either 5000 SQ, 25000 SQ or 125000 SQ for six weeks, followed by one immunization with alum-adsorbed Phl p. Spleen cells were isolated 10 days later and restimulated in vitro with Phl p. Proliferation was measured after 6 days of incubation.

Within the range of 5000-125000 SQ, the dose of Phl p used as SLIT treatment does not seem to be critical for the induction of T-cell tolerance. As seen in FIG. 8 the levels of suppression of the Phl p specific response induced by feeding 5000 SQ, 25000 SQ and 125000 SQ are similar, although there is a tendency towards a more effective suppression in mice that received 125000 SQ.

### Conclusion

The results demonstrate that SLIT treatment of naive mice has a preventive effect and that SLIT treatment primes the immune system. Furthermore, the suppression of both B and T cell responses after repeated immunizations indicate that this priming results in the induction of systemic tolerance.

1. A method of preventive treatment of allergy to an allergen in a subject comprising
a) administering an allergy vaccine containing the allergen as active substance to the subject via an oromucosal route,
b) wherein the subject to be treated is unsensitised in the sense of exhibiting no lgE response specific to the allergen,
c) wherein the subject to be treated is free of clinical symptoms of any allergy, and
d) wherein the preventive treatment is aimed at preventing or reducing subsequent clinical symptoms of the allergy associated with the allergen.

2. A method according to claim 1, wherein the subject is unsensitised in the sense of exhibiting no Th2 cell response specific to the allergen.

3. A method according to claim 1, wherein the subject is free of the clinical symptoms of rhinitis, conjunctivitis, rhinorrhea, nasal obstruction, sinusitis, sneezing, atopic dermatitis, itching, watery eyes, watery nose, wheezing, skin irritation and food allergy.

4. A method according to any of claim 1, wherein the subject is unsensitised to any allergen.

5. A method according to claim 1, wherein the subject is less than 40 years, preferably less than 30 years, more preferably less than 20 years and most preferably between 2 and 10 years of age.

6. A method according to claim 1, wherein the allergen is selected from the group consisting of an inhalation allergen and a venom allergen.

7. A method according to claim 6, wherein the allergen is selected from the group consisting of a tree pollen allergen, a grass pollen allergen, a dust mite allergen, a herb allergen and an animal allergen.

8. A method according to claim 1 comprising administering an allergy vaccine containing the allergen as active substance to the subject via a parenteral route.

9. A method according to claim 8, wherein the administration via a parenteral route is carried out subsequent to the administration via the oromucosal route.

10. A method according to claim 8, wherein the administration via a parenteral route is carried out prior to the administration via the oromucosal route.

11. Use of an allergen for the manufacture of a vaccine for the preventive treatment of allergy in a subject,
a) wherein the vaccine is suitable for administration via an oromuscosal route.
b) wherein the subject to be treated is unsensitised in the sense of exhibiting no lgE response specific to the allergen,
c) wherein the subject to be treated is free of clinical symptoms of any allergy, and
d) wherein the preventive treatment is aimed at preventing or reducing subsequent clinical symptoms of the allergy associated with the allergen.
